(19) United States
(12) Patent Application Publication
(10) Pub. No.: US 2024/0033051 A1
(43) Pub. Date: Feb. 1, 2024

(54) IMPLANT SYSTEM FOR A DENTURE

(71) Applicant: TRUABUTMENT KOREA, INC., Bucheon-si (KR)

(72) Inventors: Haeng Oh KIM, Busan (KR); Seung Hwa LEE, Gwangmyeong-si (KR)

(21) Appl. No.: 18/353,346

(22) Filed: Jul. 17, 2023

(30) Foreign Application Priority Data
Jul. 26, 2022 (KR) 10-2022-0092692

(51) Int. Cl. A61C 8/00

(52) U.S. Cl. CPC A61C 8/0066; A61C 8/0069; A61C 8/0022; A61C 8/0095; A61C 2008/0084

(57) ABSTRACT

Disclosed herein an implant system for a denture. The denture implant system includes: an abutment and a screw configured to connect the abutment to a denture. The abutment comprises: an apex portion configured to face the denture and have linear edges that are parallel at least in an intermediate area, a thread hole provided in one end area of the apex portion and configured to accommodate the screw, and a fixture hole which is spaced apart from the thread hole and is formed toward a fixture engagement portion under the apex portion.

### CROSS REFERENCE TO RELATED APPLICATION

The present application claims priority to a Korean patent application 10-2022-0092692, filed Jul. 26, 2022, the entire contents of which are incorporated herein for all purposes by this reference.

### BACKGROUND OF THE INVENTION

### Field of the invention

The present disclosure relates to an implant system for a denture, and more specifically, to an implant system for a denture for maintaining a stable fastening of a screw for engagement between the denture and an abutment, while also ensuring the convenience of temporary prosthesis procedures and the ease of fabrication of implant components.

### Description of the Related Art

In general, an implant comprises a fixture, which is implanted in a jawbone, an abutment, which is inserted and fixed to the fixture, and a prosthesis that is fixed so as to envelop the upper portion of the abutment.

A fixture, which is implanted in a jawbone, plays the role of tooth root, a prosthesis is made in a tooth-like shape to become the visible part of an artificial tooth, and an abutment fixes a prosthesis to a fixture so that loads on the prosthesis are transmitted to the fixture and the jawbone.

A prosthesis may be a crown, a bridge, or a denture. A crown may be configured as an individual artificial tooth and fixed to an abutment. A bridge may span an area of an extracted tooth and adjacent teeth in a bridge-like formation and may be fixed to the abutment. In case a considerable number of teeth in the maxilla or mandible are damaged and extracted, a denture may be fabricated to comprise a plurality of artificial teeth as a single unit for replacing the extracted teeth. Dentures may be categorized into removable and fixed denture. A removable denture may be engaged via a connecting component at a specific location within the oral cavity and may be easily detached for cleaning and maintenance purposes. A fixed denture, also referred to as an implant denture, may be securely engaged with a fixture implanted in the jawbone and an abutment, preventing separation of the denture within the oral cavity. Such fixation may be achieved through a screw connecting the denture and the abutment.

In the case of a fixed denture, various directional stresses may be applied to an abutment and the denture due to occlusal forces. The stresses may be concentrated on a screw that engages the abutment and the denture together in a fixed manner.

In addition, the abutment may be equipped with a bore for accommodating a fixture screw for securing the fixture, apart from a hole for screwing and coupling with the screw. In a conventional abutment, a hole and a bore may partially overlap in their formation, depending on a connection point with a denture and a connection point with a fixture. In this case, the hole may have a partially discontinuous thread, which is formed due to the bore, instead of a continuous threaded shape. Specifically, along a direction of the hole into which the screw is inserted, a portion of the hole may be exposed by the bore, and this part has no thread. Even when the screw is fully inserted into the hole, it does not engage with the exposed portion of the hole. As the stress-concentrated screw includes some unengaged threads, the screw may be more susceptible to stress.

Due to a stress, a screw may partially loosen, or a screw thread may be damaged, weakening the fastening force, or even causing the screw to break. As a result, not only may a denture not be stably fixed to an abutment, but a serious harmful factor may also be induced in the oral cavity.

An artificial gum and a palate/floor of a denture may be fabricated to conform to a patient's oral structure and jawbone model. Accordingly, the artificial gum and the palate/floor may be formed with various shapes and orientations along the overall contour. Meanwhile, for robust fixation, the denture may be engaged with multiple abutments in order to be connected to a plurality of fixtures implanted at various points in the jawbone. To correspond to various shapes and orientations, each abutment may be fabricated with a junction with the denture and a junction with the fixture, which have distinct shapes and orientations. As described above, although the fixture junction is constructed in a different shape, the distal ends of the junctions may, for instance, be formed with the same hexagonal size thereby ensuring no substantial difficulties in fabrication and the engagement procedure with the fixture.

However, as a denture junction of a conventional abutment and its distal end are formed in different shapes, meticulous attention concerning fabrication is required. Furthermore, in denture fabrication, an accommodating groove with a corresponding shape to each abutment's junction should be processed, and this necessitates considerable attention in denture production as well.

In addition, a scan body may be used to fabricate a personalized denture by using a CAD model. With the scan body attached to each abutment, a manufacturer may digitally verify an alignment position and orientation of the scan body and fabricate the denture. Since the denture junctions on each abutment have different shapes, the manufacturer should ensure to select a scan body that matches the shape of the abutment when choosing a scan body.

In order to fabricate a personalized denture, each abutment is formed with denture junctions of different shapes, which results in decreased convenience in the temporary prosthesis procedure and makes the fabrication of implant components less feasible.

### SUMMARY

The present disclosure is technically directed to provide an implant system for a denture for maintaining a stable fastening of a screw for engagement between the denture and an abutment, while also ensuring the convenience of temporary prosthesis procedures and the ease of fabrication of implant components.

The objects of the present disclosure are not limited to the above-mentioned objects, and other objects not mentioned can be clearly understood by those skilled in the art from the following description.

According to the present disclosure, there is provided a denture implant system, the denture implant system comprising: an abutment and a screw configured to connect the abutment to a denture. the abutment comprises an apex portion configured to face the denture and have linear edges that are parallel at least in an intermediate area, a thread hole provided in one end area of the apex portion and configured to accommodate the screw, and a fixture hole which is spaced apart from the thread hole and is formed toward a fixture engagement portion under the apex portion.

According to the embodiment of the present disclosure in the denture implant system, an other end area of the apex portion, which is disposed at an opposite side of the one end area, and the intermediate area of the apex portion may be connected with each other so as to form a track shape.

According to the embodiment of the present disclosure in the denture implant system, the other end area may be formed in an arc shape that employs a width between the linear edges as a diameter.

According to the embodiment of the present disclosure in the denture implant system, the one end area of the apex portion may have a larger width than the width between the linear edges.

According to the embodiment of the present disclosure in the denture implant system, the fixture hole may be formed spacedly apart from the apex portion or is formed to pass through a part of the apex portion. Also, the thread hole may be formed separately not to be exposed by the fixture hole when the fixture hole is formed to pass through the part of the apex portion.

According to the embodiment of the present disclosure in the denture implant system, the thread hole may be formed to have a closed cylindrical shape so that a thread in the thread hole is formed to be continuous.

According to the embodiment of the present disclosure in the denture implant system, the screw may comprise a thread portion engaged with the thread hole, a head disposed on top of the thread portion, and a taper portion that is disposed between the thread portion and the head and has an outer diameter that decreases toward the thread portion. Also, the denture may comprise a denture hole for accommodating the screw, and the denture hole has a taper side wall corresponding to the taper portion.

According to the embodiment of the present disclosure in the denture implant system, the abutment may comprise a protrusion portion and an extension portion that are disposed between the apex portion and the fixture engagement portion, the protrusion portion is formed to be extended toward the apex portion, and the extension portion is disposed under the protrusion portion and has a larger outer periphery than the protrusion portion in a lateral direction. Also, the denture may have a plurality of accommodation portions engaged with a plurality of abutments, and each of the plurality of the accommodation portions has a same shape and size in order to correspond at least to the apex portion and the protrusion portion.

According to the embodiment of the present disclosure in the denture implant system, the denture implant system may further comprise a scan body that is engaged with the abutment and has a base portion with a through hole being formed, a screw for scan configured to connect the scan body and the abutment through the through hole and the thread hole, and a temporary prosthesis that is temporarily installed by being attached to the scan body. The temporary prosthesis may have a plurality of prosthesis accommodation portions engaged with a plurality of scan bodies, and each of the plurality of the prosthesis accommodation portions may have a same shape and size in order to correspond to the through hole and the base portion.

According to the embodiment of the present disclosure in the denture implant system, the abutment may comprise a protrusion portion and an extension portion that are disposed between the apex portion and the fixture engagement portion, the protrusion portion is formed to be extended toward the apex portion, and the extension portion is disposed under the protrusion portion and has a larger outer periphery than the protrusion portion in a lateral direction. Also, the scan body may have a groove engaged with the abutment, and a groove of each of the scan body has a same shape and size in order to correspond at least to the apex portion and the protrusion portion in a case of presence of a plurality of the scan body.

According to the present disclosure, it is possible to provide an implant system for a denture for maintaining a stable fastening of a screw for engagement between the denture and an abutment, while also ensuring the convenience of temporary prosthesis procedures and the ease of fabrication of implant components.

Effects obtained in the present disclosure are not limited to the above-mentioned effects, and other effects not mentioned above may be clearly understood by those skilled in the art from the following description.

In addition, no effect is excluded which may be derived from a configuration that those skilled in the art are able to understand through the present specification.

### BRIEF DESCRIPTION OF THE DRAWINGS

FIG. 1 is a perspective view exemplifying an implant system for a denture according to the present disclosure.

FIG. 2A and FIG. 2B are a top perspective view and a front perspective view of an example of an abutment, respectively.

FIG. 3A and FIG. 3B are a top perspective view and a front perspective view of another example of an abutment, respectively.

FIG. 4A and FIG. 4B are a top perspective view and a front perspective view of yet another example of an abutment, respectively.

FIG. 5A and FIG. 5B are a front perspective view and a top perspective view respectively exemplifying a screw.

FIG. 6A and FIG. 6B are a top perspective view and a bottom perspective view respectively exemplifying a denture.

FIG. 7 is a sectional view exemplifying the coupling of an abutment and a denture through a screw.

FIG. 8A and FIG. 8B are a top perspective view and a bottom perspective view respectively exemplifying a scan body.

FIG. 9 is a perspective view of a screw for scan.

FIG. 10 is a bottom perspective view of a temporary prosthesis.

### DETAILED DESCRIPTION

Hereinafter, exemplary embodiments of the present disclosure will be described in detail with reference to the accompanying drawings so that those skilled in the art may easily implement the present disclosure. However, the present disclosure may be implemented in various different ways, and is not limited to the embodiments described therein.

In describing exemplary embodiments of the present disclosure, well-known functions or constructions will not be described in detail since they may unnecessarily obscure the understanding of the present disclosure. The same constituent elements in the drawings are denoted by the same reference numerals, and a repeated description of the same elements will be omitted.

In the present disclosure, when an element is simply referred to as being “connected to”, “coupled to” or “linked to” another element, this may mean that an element is “directly connected to”, “directly coupled to” or “directly linked to” another element or is connected to, coupled to or linked to another element with the other element intervening therebetween. In addition, when an element “includes” or “has” another element, this means that one element may further include another element without excluding another component unless specifically stated otherwise.

Hereinafter, exemplary embodiments of the present invention will be described in detail with reference to the accompanying drawings and the contents described below. However, the present invention is not limited to the embodiments described herein and may be embodied in other forms. Rather, the embodiments introduced herein are provided to ensure that the disclosed subject matter is thorough and complete, and that the scope of the invention will be fully conveyed to those skilled in the art. Like numbers refer to like elements throughout. Meanwhile, the terminology used herein aims to describe particular embodiments only and is not intended to limit the present invention. In this specification, the singular forms also include the plural unless specifically stated otherwise in the text. As used herein, “comprises” and/or “comprising” specifies the presence of the mentioned shapes, numbers, steps, actions, members and/or elements. It does not exclude the presence or addition of one or more other shapes, numbers, steps, actions, members, elements and/or groups thereof.

In the present disclosure, the terms first, second, etc. are only used to distinguish one element from another and do not limit the order or the degree of importance between the elements unless specifically mentioned. Accordingly, a first element in an embodiment could be termed a second element in another embodiment, and, similarly, a second element in an embodiment could be termed a first element in another embodiment, without departing from the scope of the present disclosure.

In the present disclosure, elements described in various embodiments do not necessarily mean essential elements, and some of them may be optional elements. Therefore, an embodiment composed of a subset of elements described in an embodiment is also included in the scope of the present disclosure. In addition, embodiments including other elements in addition to the elements described in the various embodiments are also included in the scope of the present disclosure.

The advantages and features of the present invention and the way of attaining them will become apparent with reference to embodiments described below in detail in conjunction with the accompanying drawings. Embodiments, however, may be embodied in many different forms and should not be constructed as being limited to example embodiments set forth herein. Rather, these embodiments are provided so that this disclosure will be complete and will fully convey the scope of the invention to those skilled in the art.

In the present disclosure, each of phrases such as “A or B”, “at least one of A and B”, “at least one of A or B”, “A, B or C”, “at least one of A, B and C”, “” at Each of the phrases such as “at least one of A, B or C” and “at least one of A, B, C or combination thereof” may include any one or all possible combinations of the items listed together in the corresponding one of the phrases.

In the present disclosure, expressions of location relations used in the present specification such as “upper”, “lower”, “left” and “right” are employed for the convenience of explanation, and in case drawings illustrated in the present specification are inversed, the location relations described in the specification may be inversely understood.

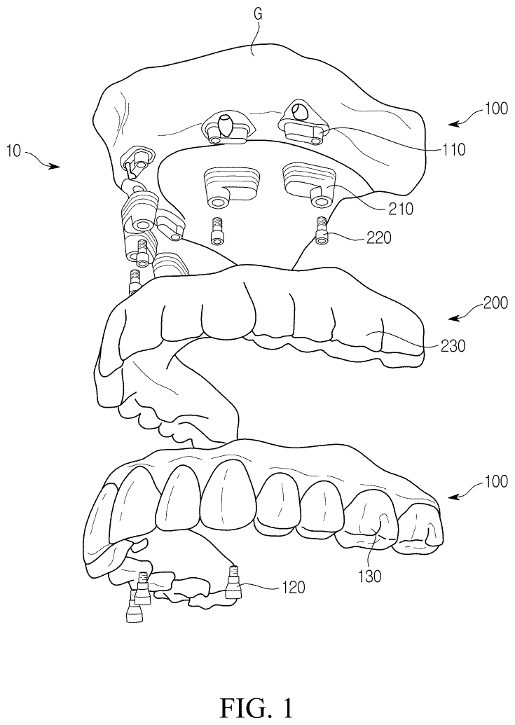

Hereinafter, referring to FIG. 1, an implant system for a denture will be described in accordance with an embodiment of the present disclosure.

FIG. 1 is a perspective view exemplifying an implant system for a denture according to the present disclosure.

An implant system 10 for a denture may comprise a denture component 100 for permanently fixing a denture 130 to a gum (G) without replacement over a long period. The denture component 100 may, for example, be referred to as an implant denture component and may include parts for constructing a fixed denture. The denture component 100 may include, for example, a plurality of fixtures (not illustrated) to be implanted and fixed at multiple sites in a jawbone within the gum G, abutments 110, screws 120, and a denture 130.

The plurality of abutments 110 may be fabricated to be fixed in fixtures. For example, an abutment 110a may be fastened to a fixture through a screw for fixture.

The denture 130 may be continuously fixed to fixtures (not illustrated) by being coupled with the abutments 110 through the plurality of the screws 120. The denture 130 is a final prosthesis used by a patient and may include artificial teeth to replace multiple extracted teeth and a supporting portion for supporting the artificial teeth. In FIG. 1, the denture 130 is exemplified as a full type of denture that replace all teeth in the upper or lower jaw, but it may also be a partial type of denture that replaces only some teeth.

In addition, the implant system 10 for a denture may further include a component 200 for scan that is used to digitally fabricate the denture 130 which is finally used.

For example, the component 200 for scan may include a scan body 210, a screw 220 for scanning, and a temporary prosthesis 230.

The scan body 210 may be connected to the plurality of the abutments (110) through the screw 120 for scanning, and a fabrication system operating a CAD model may verify an alignment position and an orientation of each scan body 210 using scanning. In order to accurately identify the position and orientation during scanning, the outer part of the scan body 210 may be formed with various forms. For example, a groove may be formed on a side of a base portion (see 211 in

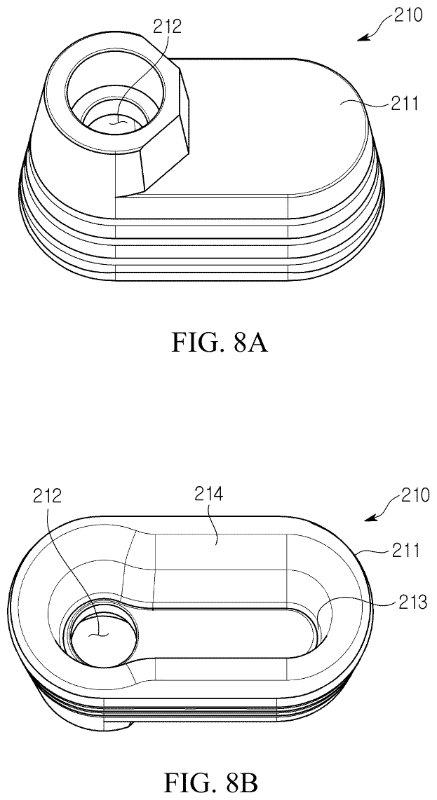

FIG. 8A) constituting the body of the scan body 210, or an area of a through hole (see 212 in FIG. 8A) that passes through the screw 120 for scanning may protrude. In addition, a cross-sectional shape of an area of the through-hole 212 area may be formed not only as a circle but also as a polygon or a combination of circles/polygons. Based on the identified position and orientation, the fabrication system may digitally design a shape applied to an entire contour of the denture 130 and fabricate the denture 130. When the denture 130 is completed and applied to a patient, the scan body 210 may be removed.

The temporary prosthesis 230 may be a false teeth that the patient uses temporarily during the fabrication process of the denture 130. When the denture 130 is completed, the temporary prosthesis 230 may be bonded to the scan body 210 using an adhesive, for example, so as to be easily separated from the scan body 210.

The implant system 10 for a denture, which includes both the denture component 100 and the component 200 for scan, may also be referred to as an implant denture system.

A more detailed description of the components constituting the denture component 100 and the component 200 for scan, which have been briefly described, will be provided.

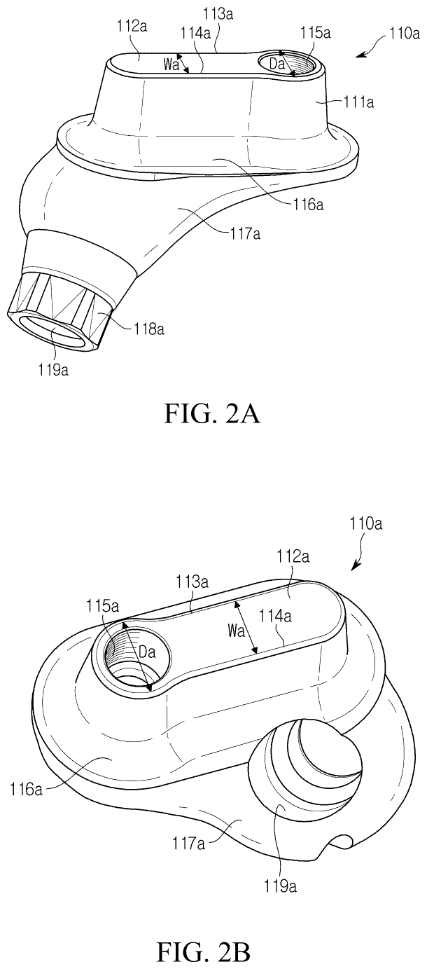

The abutment 110a of the denture component 100 may have various forms based on factors such as the patient's oral structure, a jawbone model, and a connecting direction with a fixture. Accordingly, the abutment 110a may be fabricated in the forms illustrated in FIGS. 2A to 4B, but is not limited to these examples and may be constructed in various forms. In the present disclosure, the abutment 110a may have various forms, but it may share common technical elements for procedural convenience and ease of component fabrication. In the following, elements common to various forms of the abutment 110a will be described mainly, while also describing elements that differ between embodiments.

First, referring to FIG. 2A and FIG. 2B, the abutment 110a may include a protrusion portion 111a, an apex portion 112a, an extension portion 116a, a column portion 117a, and a fixture engagement portion 118a.

The apex portion 112a is a part that faces the denture 130 or the scan body 210 during a procedure and may be formed, for example, as a flat surface. For convenience of description, the apex portion 112a will be described as a point where it faces the denture 130.

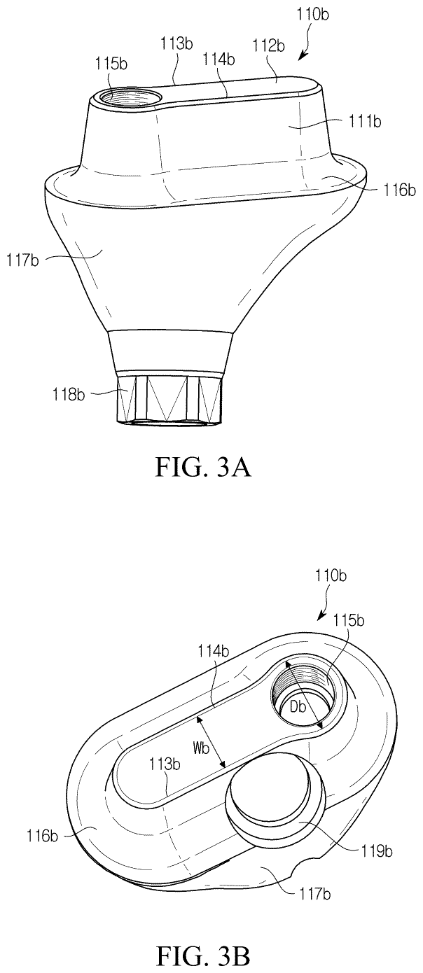

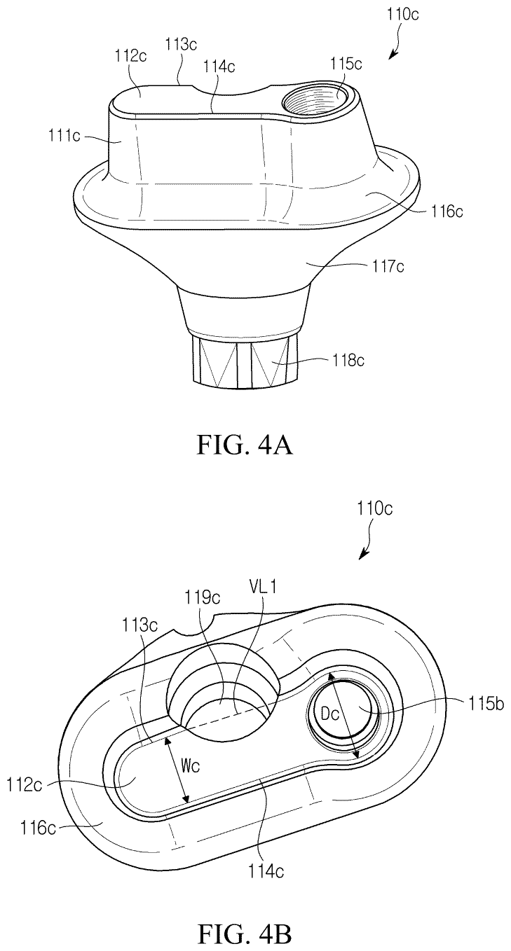

Despite the application of various forms described above, the apex portion 112a may be formed to have substantially a same shape for the convenience of temporary prosthetic procedures and the ease of component fabrication of the system 10. This may mean that the apex portion 112a of the abutments 110b and 110c according to the embodiments in FIG. 3A to FIG. 4B has a substantially identical shape to that of FIG. 2A and FIG. 2B.

The apex portion 112a may be provided with a thread hole 115a for accommodating the screw 120 in one end area. The apex portion 112a may have first and second linear edges 113a, 114a that are parallel at least in an intermediate area. Herein, the one end area of the apex portion 112a may have a width (Da) greater than a width (Wa) between the first and second linear edges 113a and 114a.

In addition, the other end area of the apex portion 112a, which is disposed on the opposite side of the one end area, may be formed as an arc shape employing the width (Wa) between the first and second linear edges 113a and 114a as its diameter. Accordingly, the other end area and the intermediate area may be connected to each other, forming at least a partial track shape in the apex portion 112a.

As the one end area has a larger width than the intermediate area and the other end area, a practitioner may easily identify a direction of the abutment 110a to be connected to a fixture. In addition, as the thread hole 115a of the one end area is formed by being relatively expanded, smooth fastening of the screw 120, stable connection with the denture 130, and robust adhesion to the denture 130 may be ensured. As the thread hole 115a and the one end area are formed to be greater than the width (Wa) between the first and second linear edges 113a and 114a, a stable connection and robust adhesion with the scan body 210 may be realized.

In addition, although other parts of the abutments 110a to 110c exemplified in FIG. 2A to FIG. 4B are constructed in different shapes, the apex portion 112a may be formed to have a substantially same shape in all the exemplified abutments 110a to 110c. For example, the other parts of the abutments 110a to 110c may be columns 117a to 117c and fixture engagement portions 118a to 118c, and these parts may have various shapes based on a patient's oral structure, a jawbone model, and a connecting direction with a fixture, as exemplified in FIG. 2A to FIG. 4B. As shown in the abutments 110a to 110c exemplified in FIG. 2A to FIG. 4B, the shape and size from an one end area to the other end area may be formed with a substantially same size in the apex portion 112a to 112c, and the shape and size of the thread holes 115a to 115c may also be formed with a substantially same size. In other words, the apex portion 112a to 112c may have a standardized shape regardless of other parts.

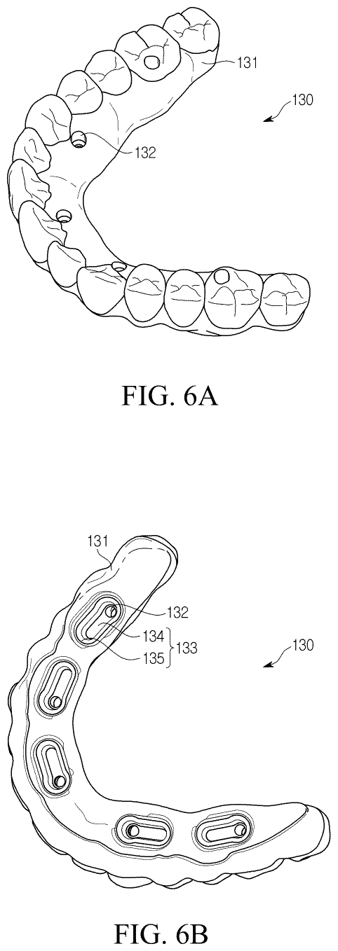

The apex portion 112a may be formed in a track shape with a same shape and size, regardless of other parts of the abutment 110a, and may also be provided with a same form of thread hole 115a, so that the apex portion 112a of the abutment 110a can be easily fabricated. In addition, as the apex portion 112a is formed in a same form of track shape and the thread hole 115a, all the accommodating spaces of the denture 130 and the scan body 210, which the apex portion 112a faces after being inserted, may be uniformly fabricated. Specifically, as shown in FIG. 6B, a plurality of accommodation portions 133 of the denture 130, where the abutments 110a to 110c with different shapes exemplified in FIG. 2A to FIG. 4B, may not be formed differently according to the shapes but be fabricated in the same shape as the apex portion 112a. In addition, the grooves 213, 214 of the plurality of scan bodies 210 accommodating each of the abutments 110a to 110c with different shapes may not be formed differently according to the shapes but be fabricated in the same shape as the apex portion 112a, as shown in FIG. 8B.

In order to form a continuous thread in the thread hole 115a, the thread hole 115a may be formed in a closed cylindrical shape. A conventional thread hole may be formed with its part being sectioned according to a position of a bore corresponding to a hole 119a for fixture to be described below. Specifically, a bore may be provided in various positions according to an orientation of an abutment and a relative position relation between a fixture and an abutment. Depending on cases, as a bore partially overlaps a thread hole and a conventional thread hole has an open cylindrical shape, the inner thread may be discontinuously formed. Because of an open cylindrical and discontinuous thread, the fastening force of the screw 120 in a conventional thread hole may be weakened. According to the present disclosure, the thread hole 115a may maintain a closed cylindrical shape by being completely spaced apart from the hole 119a for fixture. Accordingly, since it is ensured that the inner thread of the thread hole 115a is continuous, the fastening force of the screw 120 may be strengthened.

The protrusion portion 111a may be extended toward the apex portion 112a. In the abutments 110a to 110c exemplified in FIG. 2A to FIG. 4B, the protrusion portion 111a may be constructed to have a substantially same shape and size, like the apex portion 112a. That is, the protrusion portions 111a to 111c may have a standardized shape irrespective of a same component that is implemented in shapes different from each other in FIG. 2A to FIG. 4B. Accordingly, the protrusion portions 111a to 111c of the abutments 110a to 110c may be easily fabricated. In addition, as the protrusion portion 111a is formed in a same shape, the denture 130, into which the protrusion portion 111a is inserted, and the accommodating spaces of the scan bodies 210 may all be uniformly fabricated. Accordingly, the fabrication of the denture 130 and the scan body 210 may be simplified.

The extension portion 116a is disposed under the protrusion portion 111a and may be constructed to have a larger outer periphery than the protrusion portion 111a in the lateral direction.

When being coupled with the denture 130, the extension portion 116a may be accommodated into the accommodation portion 133, for example, the outer groove 135, and thus contribute to the stable insertion and firm fixation of the abutment 110a into the denture 130. In addition, when being coupled with the scan body 210, the extension portion 116a may be accommodated into a groove of the scan body 210, for example, the outer groove 135, and thus the stable insertion and firm fixation of the abutment 110a into the scan body 210 may be realized.

The hole 119a for fixture may be a hole into which a screw for fixture (not illustrated) to be fastened with a fixture is inserted. The hole 119a for fixture may be spaced from the thread hole 115a and be formed toward the fixture engagement portion 118a. As described above, the hole 119a for fixture may be provided in various positions according to an orientation of the abutment 110a and a relative position relation with a fixture. A relative position with a fixture may be determined according to a direction and position in which the abutment 110a is connected to a fixture. In FIG. 2A and FIG. 2B, the hole 119a for fixture is exemplified to be formed spacedly apart from the apex portion 112a. In this example, the hole 119a for fixture is formed to pass through the extension portion 116a and the column portion 117a and to be extended to the fixture engagement portion 118a. As the hole 119a for fixture does not overlap the thread hole 115a, the thread hole 115a may be formed not to be exposed by the hole 119a for fixture. Furthermore, an inner thread of the thread hole 115a may be maintained to be continuous.

The column portion 117a may be disposed under the extension portion 116a, and the fixture engagement portion 118a may be formed under the column portion 117a. As described above, the column portion 117a may have various shapes based on a patient's oral structure, a jawbone model, and a connecting direction with a fixture. For example, based on the above-described condition, the column portion 117a may be processed at a suitable angle from the fixture engagement portion 118a, and a body structure of the column portion 117a may also be formed to have an optimal shape according to the condition.

In order to be inserted into a head of a fixture, the fixture engagement portion 118a may be formed to have a corresponding structure to the shape of the head. By using a procedure tool, a thread of a screw for fixture may be advanced to the outside of the fixture engagement portion 118a and be fastened with an inner thread of a fixture. According to a relative position relation with the column portion 117a, the fixture engagement portion 118a may be disposed at a predetermined angle for the column portion 117a.

Apart from the abutment 110a according to the embodiments of FIG. 2A and FIG. 2B, the abutment 110a according to the embodiments of FIG. 3A to FIG. 4B will be described.

FIG. 3A and FIG. 3B are a top perspective view and a front perspective view of another example of an abutment, respectively.

Like the embodiments of FIG. 2A and FIG. 2B, the abutment 110b may include the protrusion portion 111b, the apex portion 112b, the thread hole 115b, the extension portion 116b, the column portion 117b, and the fixture engagement portion 118b. As the above-described components are substantially the same as described in FIG. 2A and FIG. 2B, no further detailed description will be provided, and the description below will focus on a difference from the embodiments of FIG. 2A and FIG. 2B.

For the convenience of temporary prosthesis procedure and the ease of fabricating components of the system 10, the apex portion 112b may be formed to have a substantially same shape as in FIG. 2A and FIG. 2B.

The apex portion 112b may have the thread hole 115b for accommodating the screw 120 in one end area. The apex portion 112b may have first and second linear edges 113b and 114b that are parallel at least in an intermediate area. Herein, the one end area of the apex portion 112b may have a width (Db) greater than a width (Wb) between the first and second linear edges 113b and 114b.

In addition, the other end area of the apex portion 112b, which is disposed at the opposite side of the one end area, may be formed as an arc shape employing the width (Wb) between the first and second linear edges 113b and 114b as its diameter. Accordingly, the other end area and the intermediate area may be connected to each other, forming at least a partial track shape in the apex portion 112b. In addition, the apex portion 112b may have a standardized shape regardless of other parts of the abutment 110b.

In order to form a continuous thread in the thread hole 115b, the thread hole 115b may be formed in a closed cylindrical shape. The protrusion portion 111b may be extended toward the apex portion 112b. The protrusion portion 111b may be constructed to have a substantially same shape and size as the apex portion 112a of FIG. 2A and FIG. 2B. The protrusion portion 111b may have a standardized shape irrespective of a same component that is implemented in shapes different from each other in FIG. 2A to FIG. 4B. The extension portion 116b is disposed under the protrusion portion 111b and may be constructed to have a larger outer periphery than the protrusion portion 111b in the lateral direction.

The hole 119b for fixture may be spaced from the thread hole 111b and be formed toward the fixture engagement portion 118b. Unlike in FIG. 2A and FIG. 2B, the hole 119b for fixture in FIG. 3A and FIG. 3B may be formed to enter a part of the protrusion portion 111b. Even when the hole 119b for fixture is provided in the protrusion portion 111b adjacent to the thread hole 115b, since the apex portion 112b is formed in a track shape, a position of the hole 119b for fixture may have more degrees of freedom than a conventional one. Accordingly, since the hole 119b for fixture does not overlap the thread hole 115b, the thread hole 115b may be formed not to be exposed by the hole 119b for fixture. Furthermore, an inner thread of the thread hole 115b may be maintained to be continuous.

The column portion 117b may be disposed under the extension portion 116b, and the fixture engagement portion 118b may be formed under the column portion 117b. As described above, the column portion 117b may have various shapes based on a patient's oral structure, a jawbone model, and a connecting direction with a fixture. The column portion 117b and the fixture engagement portion 118b may be fabricated to an optimal shape and orientation different from those of FIG. 2A and FIG. 2B, based on the above-described condition and their relative position relation.

FIG. 4A and FIG. 4B are a top perspective view and a front perspective view of yet another example of an abutment, respectively.

Like the embodiments of FIG. 2A and FIG. 2B, the abutment 110c may include the apex portion 112c, the thread hole 115c, the protrusion portion 111c, the extension portion 116c, the column portion 117c, and the fixture engagement portion 118c. As the above-described components are substantially the same as described in FIG. 2A and FIG. 2B, no further detailed description will be provided, and the description below will focus on a difference from the embodiments of FIG. 2A and FIG. 2B.

For the convenience of temporary prosthesis procedure and the ease of fabricating components of the system 10, the apex portion 112c may be formed to have a substantially same shape as in FIG. 2A and FIG. 2B.

The apex portion 112c may be provided with a thread hole 115c for accommodating the screw 120 in one end area. The apex portion 112c may have first and second linear edges 113c and 114c that are parallel at least in an intermediate area. The one end area of the apex portion 112c may have a width (Dc) greater than a width (Wc) between the first and second linear edges 113c and 114c.

Herein, unlike FIG. 2A and FIG. 2B, the hole 119c for fixture may be formed to pass through a part of the apex portion 112c. For this reason, the first linear edge 113c of the apex portion 112c may have a discontinuous (or interrupted) linear form. The first linear edge 113c may be fabricated to have a single line when being connected with a virtual line VL1 corresponding to a discontinuous part. Despite being a discontinuous linear form, the first linear edge 113c may be formed to have a parallel line to the second linear edge 114c. In addition, the other end area of the apex portion 112c, which is disposed on the opposite side of the one end area, may be formed as an arc shape employing the width (Wc) between the first and second linear edges 113c and 114c as its diameter. The other end area and the intermediate area may be connected to each other, forming at least a partial track shape in the apex portion 112c. As the apex portion 112c is formed in a substantially track shape, the benefits and effects described in FIG. 2A and FIG. 2B may be made likewise. In addition, the apex portion 112c may have a standardized shape regardless of other parts of the abutment 110c.

In order to form a continuous thread in the thread hole 115c, the thread hole 115c may be formed in a closed cylindrical shape. The protrusion portion 111c may be extended toward the apex portion 112c. The protrusion portion 111c may be constructed to have a substantially same shape and size as the apex portion 112a of FIG. 2A and FIG. 2B. The protrusion portion 111c may have a standardized shape irrespective of a same component that is implemented in shapes different from each other in FIG. 2A to FIG. 4B. The extension portion 116c is disposed under the protrusion portion 111c and may be constructed to have a larger outer periphery than the protrusion portion 111c in the lateral direction.

The hole 119c for fixture may be spaced from the thread hole 115c and be formed toward the fixture engagement portion 118c. Unlike in FIG. 2A and FIG. 2B, the hole 119c for fixture in

FIG. 4A and FIG. 4B may be formed to pass through the protrusion portion 111c up to a part of the apex portion 112c. Even when the hole 119c for fixture is provided in the apex portion 112c adjacent to the thread hole 115c, since the apex portion 112c is formed in a track shape, a position of the hole 119c for fixture may have more degrees of freedom than a conventional one. Accordingly, since the hole 119c for fixture does not overlap the thread hole 115c, the thread hole 115a may be formed not to be exposed by the hole 119c for fixture. Furthermore, an inner thread of the thread hole 115c may be maintained to be continuous.

Unlike the examples of FIG. 4A and FIG. 4B, the hole 119c for fixture may be formed to enter an arc corresponding to the other end area of the apex portion 112c. In this case, the first and second linear edges 113c and 114c may be arranged as lines parallel to each other. In addition, when discontinuous (or interrupted) points are connected through a virtual line, a discontinuous arc may have a substantially same shape and size as in FIG. 2A and FIG. 2B. In addition, an arc including a virtual line may be fabricated to be connected with the first and second linear edges 113c and 114c, and thus the apex portion 112c may have a track shape.

The column portion 117c may be disposed under the extension portion 116c, and the fixture engagement portion 118c may be formed under the column portion 117c. As described above, the column portion 117c may have various shapes based on a patient's oral structure, a jawbone model, and a connecting direction with a fixture. The column portion 117c and the fixture engagement portion 118c may be fabricated to an optimal shape and orientation different from those of FIG. 2A and FIG. 2B, based on the above-described condition and their relative position relation.

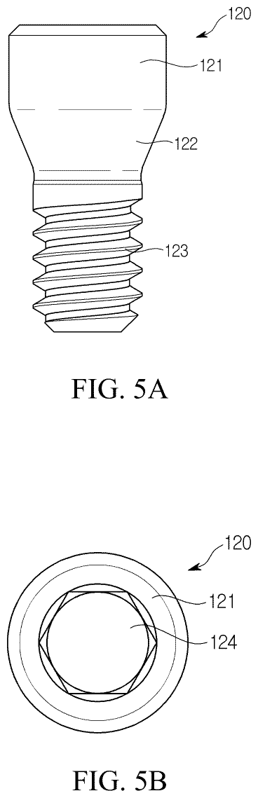

In FIG. 5A to FIG. 10 below, the components of the abutment 110a will be described through the reference symbols of FIG. 2A and FIG. 2B. However, the reference symbols of FIG.

3A to FIG. 4B are omitted for convenience of description but may also be applied to the abutments 110b, 110c of FIG. 3A to FIG. 4B.

FIG. 5A and FIG. 5B are a front perspective view and a top perspective view respectively exemplifying a screw.

The screw 120 may be a component that connects the abutment 110a to the denture 130. The screw 120 may include a head 121, a thread portion 123, which is disposed under the head 121 and is to be connected to the thread hole 115a, and a taper portion 122 that is disposed between the head 121 and the thread portion 123.

For example, the thread portion 123 is a part that is fastened to the thread hole 115a during the connection between the abutment 110a and the denture 130, and the head 121 may have, for example, a tool settlement portion 124 in which a procedure tool is inserted in the connection.

The taper portion 122 may be formed as a sloped outer periphery that has a gradually decreasing outer diameter toward the thread portion 123. The taper portion 122 may be fabricated to have an outer periphery corresponding to a taper side wall 137 of a denture hole 132 in the denture 130.

FIG. 6A and FIG. 6B are a top perspective view and a bottom perspective view respectively exemplifying a denture.

For example, the denture 130 may include a body 131 supporting a plurality of artificial teeth, a denture hole 132 in which the screw 120 is inserted to connect/fix the denture 130 to the abutment 110a, and an accommodation portion 133 that accommodates the abutment 110a in the connection/fixation.

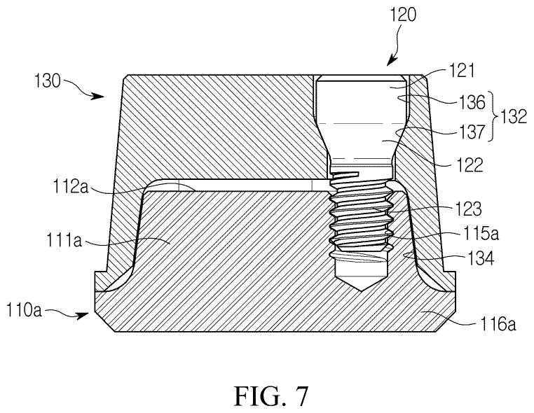

As exemplified in FIG. 7, the denture hole 132 may be constructed to include a linear side wall 136 corresponding to the head 121 of the screw 120 and the taper side wall 137 corresponding to the taper portion 122 of the screw 120. The taper side wall 137 may slope to have a small inner diameter toward the accommodation portion 133.

FIG. 7 is a sectional view exemplifying the coupling of an abutment and a denture through a screw.

In order to permanently fix the denture 130 to the abutment 110a, a practitioner may fasten the screw 120 to the thread hole 115a through the denture hole 132. After the thread hole 115a is completely fastened, various directional stresses may be concentrated to the screw 120 due to a patient's occlusal force. However, as the taper portion 122 of the screw 120 and the taper side wall 137 of the denture 130 support each other in contact, the concentrated stresses may be dispersed or mitigated. Accordingly, the loosening and damage of the fastened screw may be prevented.

The accommodation portion 133 may have an inner groove 134, which accommodates the apex portion 112a and the protrusion portion 111a of the abutment 110a, when being connected with the abutment 110a, and an outer groove 135 that accommodates the extension portion 116a. As described above, since the apex portion 112a and the protrusion portion 111a are fabricated in a same shape and size in the abutment 110a with various shapes, a plurality of the inner grooves 134 may be uniformly fabricated, thereby enhancing the convenience of fabricating the denture 130. In addition, as the extension portion 116a contacting at least the outer groove 135 is also fabricated in a same shape and size in the abutment 110a with various shapes, a plurality of the accommodation portion 133 may be easily fabricated.

FIG. 8A and FIG. 8B are a top perspective view and a bottom perspective view respectively exemplifying a scan body.

The scan body 210 may have the base portion 211, which is used to identify a position and an orientation in scanning, the through hole 212 formed in the base portion 211, and grooves 213, 214 that accommodate the abutment 110a in temporary connection/fixation to the abutment 110a.

The grooves 213, 214 may be provided, and the inner groove 213 accommodates the apex portion 112a and the protrusion portion 111a of the abutment 110a, and the outer groove 214 accommodates the extension portion 116a, when the grooves are engaged with the abutment 110a. As described above, since the apex portion 112a and the protrusion portion 111a are fabricated in a same shape and size in the abutment 110a with various shapes, the inner groove 213 is fabricated uniformly for a plurality of scan bodies, thereby enhancing the convenience of fabricating the scan body 210. In addition, as the extension portion 116a contacting at least the outer groove 214 is also fabricated in a same shape and size in the abutment 110a with various shapes, a groove applied to a plurality of the scan bodies 210 may be fabricated uniformly and easily.

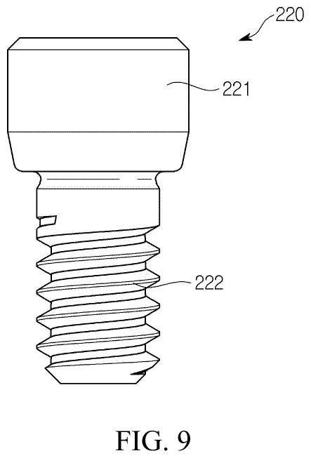

As exemplified in FIG. 1, the screw 120 for scanning may be used to fix the scan body 210 to the abutment 110a for the purpose of scanning. As exemplified in FIG. 9, the screw 120 for scanning may have a head 221 for settling a procedure tool and a thread portion 222 to be fastened in the thread hole 115a of the abutment 110a. FIG. 9 is a perspective view of a screw for scan. Apart from the shape exemplified in FIG. 9, an outer wall of the head 221 may be formed like the screw 120 of FIG. 5A and FIG. 5B, but is not limited thereto and may be processed in various shapes.

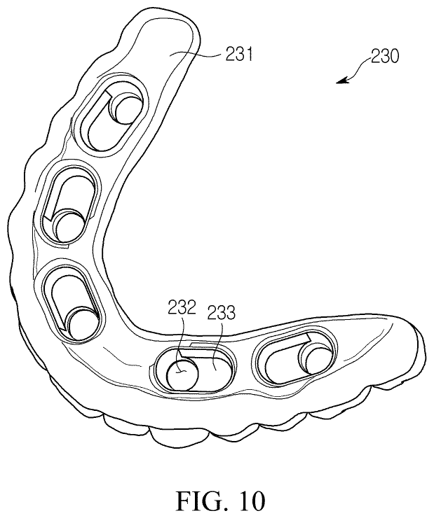

FIG. 10 is a bottom perspective view of a temporary prosthesis.

The temporary prosthesis 230 may have the body 131 supporting temporary teeth, a prosthesis hole 232 for accommodating the through hole 212 of the scan body 210, and a prosthesis recess 233 to be connected with the base portion 211 of the scan body 210. The prosthesis hole 232 and the prosthesis recess 233 may constitute a prosthesis accommodation portion. As the abutment 110a is standardized from the apex portion 112a to the extension portion 116a, a plurality of the scan bodies 210 may be fabricated in a same shape and size according to the standardized abutment 110a. Thus, a plurality of prosthesis accommodation portion 233 may be fabricated to have a same shape and size due to the standardized through hole 212 and base portion 211 of the scan body 210. In other words, the convenience of fabricating the temporary prosthesis 230 may be improved.

Although the present invention has been described in detail through the representative embodiments above, it will be understood by those skilled in the art that various modifications can be made without departing from the scope of the present invention with respect to the above-described embodiments. Therefore, the scope of the present invention should not be limited to the embodiments described, but should be defined by all changes or modifications derived from the claims and the equivalent concepts thereto.

What is claimed is:

1. A denture implant system, comprising:
an abutment; and
a screw configured to connect the abutment to a denture,
wherein the abutment comprises:
an apex portion configured to face the denture and have linear edges that are parallel at least in an intermediate area;
a thread hole provided in one end area of the apex portion and configured to accommodate the screw; and
a fixture hole which is spaced apart from the thread hole and is formed toward a fixture engagement portion under the apex portion.

2. The denture implant system of claim 1, wherein an other end area of the apex portion, which is disposed at an opposite side of the one end area, and the intermediate area of the apex portion are connected with each other so as to form a track shape.

3. The denture implant system of claim 2, wherein the other end area is formed in an arc shape that employs a width between the linear edges as a diameter.

4. The denture implant system of claim 2, wherein the one end area of the apex portion has a larger width than the width between the linear edges.

5. The denture implant system of claim 1, wherein the fixture hole is formed spacedly apart from the apex portion or is formed to pass through a part of the apex portion, and
wherein the thread hole is formed separately not to be exposed by the fixture hole when the fixture hole is formed to pass through the part of the apex portion.

6. The denture implant system of claim 1, wherein the thread hole is formed to have a closed cylindrical shape so that a thread in the thread hole is formed to be continuous.

7. The denture implant system of claim 1, wherein the screw comprises a thread portion engaged with the thread hole, a head disposed on top of the thread portion, and a taper portion that is disposed between the thread portion and the head and has an outer diameter that decreases toward the thread portion, and
wherein the denture comprises a denture hole for accommodating the screw, and the denture hole has a taper side wall corresponding to the taper portion.

8. The denture implant system of claim 1, wherein the abutment comprises a protrusion portion and an extension portion that are disposed between the apex portion and the fixture engagement portion, the protrusion portion is formed to be extended toward the apex portion, and the extension portion is disposed under the protrusion portion and has a larger outer periphery than the protrusion portion in a lateral direction, and
wherein the denture has a plurality of accommodation portions engaged with a plurality of abutments, and each of the plurality of the accommodation portions has a same shape and size in order to correspond at least to the apex portion and the protrusion portion.

9. The denture implant system of claim 1, further comprising:
a scan body that is engaged with the abutment and has a base portion with a through hole being formed;
a screw for scan configured to connect the scan body and the abutment through the through hole and the thread hole; and
a temporary prosthesis that is temporarily installed by being attached to the scan body,
wherein the temporary prosthesis has a plurality of prosthesis accommodation portions engaged with a plurality of scan bodies, and each of the plurality of the prosthesis accommodation portions has a same shape and size in order to correspond to the through hole and the base portion.

10. The denture implant system of claim 9, wherein the abutment comprises a protrusion portion and an extension portion that are disposed between the apex portion and the fixture engagement portion, the protrusion portion is formed to be extended toward the apex portion, and the extension portion is disposed under the protrusion portion and has a larger outer periphery than the protrusion portion in a lateral direction, and
wherein the scan body has a groove engaged with the abutment, and a groove of each of the scan body has a same shape and size in order to correspond at least to the apex portion and the protrusion portion in a case of presence of a plurality of the scan body.
